(19) United States
(12) Patent Application Publication
(10) Pub. No.: US 2006/0129002 A1
(43) Pub. Date: Jun. 15, 2006

(54) Process for the synthesis of alkylresorcinols

(75) Inventors: Suzanne Wassmann-Wilken, Hannover (DE); Hans-Jurgen Schmidt, Seelze Ot Lohnde (DE); Jorg Wilken, Hannover (DE); Andreas Kanschik-Conradsen, Garbsen (DE)

Correspondence Address: COLLEEN D. SZUCH;Honeywell International Inc.-Patent Department, 101 Columbia Road, Morristown, NJ, 07962, US

(73) Assignee: Honeywell International Inc.

(21) Appl. No.: 11/011,531

(22) Filed: Dec. 15, 2004

(51) Int. Cl. C07C 39/10

(52) U.S. Cl. 568/763

(57) ABSTRACT

The invention provides a process for the production of 4-(C₂-C₁₆)alkylresorcinol. 4-(C₂-C₁₆)alkylresorcinol is produced by reacting resorcinol with a (C₂-C₁₆)alkyloic acid in the presence of a zinc chloride catalyst to produce an intermediate comprising a 4-(C₂-C₁₆)acylresorcinol and zinc ions. Then the intermediate is adjusted such that the zinc ion content is from about 0.1 to about 150 parts per million based on the weight of the intermediate to form an adjusted intermediate. Then the produced 4-(C₂-C₁₆)acylresorcinol is hydrogenated in the presence of the zinc ions and a base metal hydrogenation catalyst to produce a crude product comprising 4-(C₂-C₁₆)alkylresorcinol, which may thereafter be isolated.

### BACKGROUND OF THE INVENTION

The present invention relates to alkylresorcinol, or more particularly to a process for the production of 4-(C₂-C₁₆)alkylresorcinol. The alkylresorcinols are useful as chemical intermediates, corrosion inhibitors, skin depigmentation agents, antiseptics, photographic chemicals and as components of epoxy resins and polymers.

It is known in the art that acylresorcinols like propionylresorcinol and caproylresorcinol can be synthesized from resorcinol by either a Friedel-Crafts-Acylation with the correspondent acid chloride or acid anhydride and a Friedel-Crafts-catalyst like AlCl₃ in a suitable solvent or using the correspondent free acid and a suitable catalyst like HF or ZnCl₂ (Nencki-reaction). The workup is mostly done aqueous with subsequent removal of solvent, where necessary, and distillation and/or crystallization. This may be seen schematically as follows:

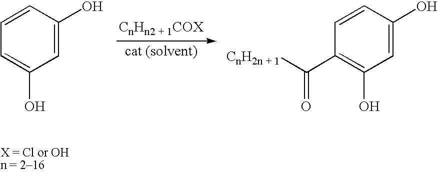

The acylresorcinols can then be hydrogenated, using either hydrogen and a suitable catalyst or other suitable hydrogenation conditions like amalgamated zinc/hydrochloric acid as seen schematically as follows. The workup is mostly distillation/crystallization, sometimes chromatography is used.

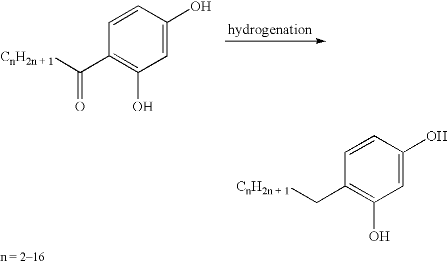

U.S. Pat. No. 4,086,281 shows that mono-substituted alkylresorcinol isomers, may be produced by reacting resorcinol and an aliphatic alcohol having 1-3 carbon atoms in the vapour phase in the presence of an acidic catalyst and by separating the resulting mono-substituted alkylresorcinol isomers from other alkylated hydroxyaromatic compounds and unreacted resorcinol. U.S. Pat. No. 4,959,393 shows the production of alkylresorcinol derivatives by a method wherein a saturated carboxylic acid and resorcinol are condensed in the presence of zinc chloride and the resultant condensate is reduced with zinc amalgam/hydrochloric acid. British Patent No. 1,581,428 shows a method wherein resorcinol and a corresponding alkyl alcohol are reacted in the presence of an alumina catalyst at a high temperature of from 200° C. to 400° C.

German patent DE489117C1 and French patent FR574131A disclose the synthesis of acylresorcinols and alkylresorcinols using a Nencki-type reaction starting from the corresponding acids, resorcinols and ZnCl₂. The acid is used as solvent. Caproylresorcinol is described as example, using a distillation/crystallization protocol as isolation procedure. Other authors use AlCl₃/acid chloride for the synthesis of acylresorcinols, but the needed solvents, e.g. nitrobenzene, 1,2-dichloroethane are not favorable in upscaled production processes. Trials to use dichlorobenzene instead give very viscous mixtures and bad yields. For a review of these techniques, see for example Houben Weyl, Volume 7/2a, part 1, (Thieme, 1973); A. K. P. Poonam et al, Tetrahedron 57 (2001), 7395-7402; J. Beger et al, J. Prakt. Chem. 334 (1992), 269-277; J. Szydlowska et al, Mol. Cryst. and Liq. Cryst. 365 (2001), 107-115.

For the hydrogenation of the acylresorcinols, in some cases amalgamated Zn/hydrochloric acid and other HgCl₂/Zn/HCl systems are used (see DE489117C1). This is not a contemporary method, and the workup is somewhat laborious, requiring an aqueous wash, various distillations, crystallization. The use of several Na-Borohydride reducing agents like NaBH₄ or NaBH₃CN is not always practicable on a large scale (e.g. C. A. Elliger, Synth. Commun. (1985), 1315-1324). A Pd/C catalyst has also been used (W. H. Hartung et al, J. Amer. Chem. Society, 56 (1934), 158 ff), but noble metal catalysts are more expensive. U.S. Pat. No. 4,419,529 describes the synthesis of alkylresorcinols from acylresorcinols using metal catalysts and hydrogen under pressure. However, trials using the hydrogenation conditions for propionylresorcinol and caproylresorcinol as described in U.S. Pat. No. 4,419,529 showed that the quality of the crude material described cannot be reached, which may be due to an insufficient analytical method used in the patent. So far undetected by-products could also account for diminished yields. More yield is lost during distillation of the alkylresorcinol, as resorcinol is formed under the distillation conditions. The hydrogenation process described in U.S. Pat. No. 4,419,529 is normally performed with acylresorcinols derived with a HF-catalyzed acylation procedure. It has now been discovered that hydrogenations of acylresorcinols with base metal catalysts like Ni, Co, and Cu where the acylresorcinols were derived from a ZnCl₂-mediated acylation process proceed under milder reaction conditions than described in U.S. Pat. No. 4,419,529. The crude products are cleaner, the process is performed at lower temperatures and pressures. The reaction runs swiftly with a sharp end point. The isolated yield is better than hydrogenations of acylresorcinols from an HF-process. Avoiding the HF-technology in the two-step sequence from resorcinol to alkylresorcinols would be an great improvement for the safety of a large scale process. Distillation can be avoided due to crude product quality, which is preferable as distillation leads to formation of resorcinol.

It has now been found that 4-(C₂-C₁₆)alkylresorcinol may be produced under more favorable conditions by reacting resorcinol with an alkyloic acid using a zinc chloride catalyst to produce an intermediate acylresorcinol and zinc ions. The zinc ion content of the intermediate is then adjusted to from about 1 to about 150 parts per million based to form an adjusted intermediate. The acylresorcinol is the hydrogenated in the presence of the zinc ions and a base metal hydrogenation catalyst to produce an alkylresorcinol crude product which may then be isolated.

### DESCRIPTION OF THE INVENTION

The invention provides a process for the production of 4-(C₂-C₁₆)alkylresorcinol which comprises reacting resorcinol with a (C₂-C₁₆)alkyloic acid in the presence of a zinc chloride catalyst to produce an intermediate comprising a 4-(C₂-C₁₆)acylresorcinol and zinc ions; adjusting the intermediate such that the zinc ion content is from about 0.1 to about 150 parts per million based on the weight of the intermediate to form an adjusted intermediate; and hydrogenating the produced 4-(C₂-C₁₆)acylresorcinol in the presence of the zinc ions and a base metal hydrogenation catalyst to produce a crude product comprising 4-(C₂-C₁₆)alkylresorcinol.

The first step in the inventive process comprises reacting resorcinol with a (C₂-C₁₆)alkyloic acid in the presence of a zinc chloride catalyst to produce an intermediate comprising a 4-(C₂-C₁₆)acylresorcinol and zinc ions. Useful (C₂-C₁₆)alkyloic acid include ethanoic acid, propionic acid, butanoic acid, pentanoic acid, hexanoic acid, heptanoic acid, octanoic acid, nonoic acid, decanoic acid, undecanoic acid, dodecanoic acid, tridecanoic acid, tetradecanoic acid, pentadecanoic acid and hexadecanoic acid. Preferred (C₂-C₁₆)alkyloic acids include propionic acid, and hexanoic acid (caproic acid).

The resorcinol may be present in the reaction mass in an amount of from about 20 weight percent to about 30 weight percent, preferably from about 20 weight percent to about 28 weight percent and more preferably from about 23 weight percent to about 25 weight percent. The (C₂-C₁₆)alkyloic acid may be present in the reaction mass in an amount of from about 55 weight percent to about 75 weight percent, preferably from about 60 weight percent to about 70 weight percent and more preferably from about 63 weight percent to about 65 weight percent. The zinc chloride may be present in the reaction mass in an amount of from about 10 weight percent to about 20 weight percent, preferably from about 12 weight percent to about 18 weight percent and more preferably from about 14 weight percent to about 16 weight percent.

The resorcinol may be reacted with the (C₂-C₁₆)alkyloic acid at a temperature of from about 90° C. to about 125° C., preferably from about 110° C. to about 125° C., and more preferably from about 117° C. to about 120° C. The resorcinol may be reacted with the (C₂-C₁₆)alkyloic acid for from about 3 hours to about 10 hours, preferably for from about 3 hours to about 5 hours, and more preferably for from about 3 hours to about 4 hours.

The resulting intermediate is adjusted such that the zinc ion content is from about 0.1 to about 150 parts per million based on the weight of the intermediate to form an adjusted intermediate. Preferably the adjusted intermediate has a zinc ion content of from about 1 to about 150 parts per million based on the weight of the intermediate to form an adjusted intermediate. More preferably the adjusted intermediate has a zinc ion content of from about 3 to about 100 parts per million based on the weight of the intermediate to form an adjusted intermediate. Still more preferably the adjusted intermediate has a zinc ion content of from about 3 to about 50 parts per million based on the weight of the intermediate to form an adjusted intermediate. The zinc ion content may be adjusted by washing with aqueous solutions (e.g. an acidic solution) and, if necessary repeated, and crystallization from suitably inert solvents such as benzene or toluene.

The produced 4-(C₂-C₁₆)acylresorcinol in the adjusted intermediate is then hydrogenated in the presence of the zinc ions and a base metal hydrogenation catalyst to produce a crude product comprising 4-(C₂-C₁₆)alkylresorcinol. Hydrogenation of the 4-(C₂-C₁₆)acylresorcinol may be conducted by reaction in a suitable solvent. Generally suitable are all solvents inert to reductive reaction conditions such as polar aprotic solvents, for example, dimethylformamide, and n-methylpyrrolidone, non-polar aliphatic and aromatic, substituted and unsubstituted solvents (e.g. toluene, chlorobenzene) but most preferably alcohols such as methanol, ethanol etc., water and also aliphatic carboxylic acids and their esters (like ethyl acetate). The base metal hydrogenation catalyst may comprise nickel, cobalt, copper or combinations thereof. Preferably the base metal catalyst comprises nickel. The hydrogenating may be conducted at a temperature of from about 20° C. to about 120° C., preferably from about 50° C. to about 90° C., and more preferably from about 60° C. to about 80° C. The hydrogenating may be conducted at a hydrogen gas pressure of from about 1 bar to about 50 bar, preferably from about 1 bar to about 30 bar, and more preferably from about 2 bar to about 20 bar. The hydrogenating may be conducted for from about 1 hour to about 10 hours, preferably from about 1 hour to about 5 hours and more preferably from about 1 hour to about 3 hours. Hydrogenation is performed in a pressure reactor (autoclave), using the above described hydrogen gas pressure, reaction time and temperature.

The crude product at this stage usually comprises 4-(C₂-C₁₆)alkylresorcinol at a purity of at least about 92%, more usually from about 92% to about 95%. This is a distinct improvement compared to hydrogenations of acylresorcinols that were not synthesized via the ZnCl₂-process, for example the hydrogen fluoride process, where the quality of the crude hydrogenated material ranges from 64-86%.

Subsequently the 4-(C₂-C₁₆)alkylresorcinol may be isolated from the crude product. 3. The isolating of the 4-(C₂-C₁₆)alkylresorcinol from the crude product may be conducted by one or more steps such as filtering, distilling, washing, crystallizing, and drying, as are well known in the art. e.g. performing a solvent exchange from alcohol to a suitable solvent like toluene, drying the solution azeotropically, letting the 4-(C₂-C₁₆)alkylresorcinol crystallize on standing, filtering off the product, washing with more solvent and drying to achieve pure 4-(C₂-C₁₆)alkylresorcinol.

The following non-limiting examples serve to illustrate the invention.

### EXAMPLE 1

### Synthesis of 4-propionylresorcinol

24.7 g ZnCl₂ are dissolved in 107.5 g of propionic acid. The mixture is heated to 125° C., and 40 g of resorcinol is added in portions. The solution is stirred 4-5 hours at 125° C. The mixture is cooled slowly and poured onto 300 mL water at 0° C. The product is filtered off and dissolved in 250 mL toluene at 80° C. The solution is washed once with 3% HCl and twice with water, and the mixture is dried azeotropically. Upon drying, the mixture is allowed to cool, the product precipitates out and is collected by filtration. The filter cake is washed twice with toluene and dried. Yield is 42%.

### EXAMPLE 2

### Synthesis of 4-propylresorcinol

100 g 4-Propionylresorcinol are dissolved in 100 mL MeOH. 30 g Ni catalyst are added and the mixture is hydrated at 60-80° C. and 10-20 bar in a suitable autoclave. The gas chromatography (GC) quality of the crude material is >92%. The catalyst is filtered off, most of the MeOH is distilled off, 400 mL of toluene are added and the mixture is distilled until the head temperature indicates full removal of the MeOH. The mixture is cooled to room temperature, and the product is filtered off. After washing with toluene and drying, the yield is 81% of 4-propylresorcinol of 99+% purity.

### EXAMPLE 3

### Synthesis of 4-caproylresorcinol

61 g ZnCl₂ are dissolved in 307 g caproic acid at 135° C. 102 g resorcinol are added in portions. The mixture is stirred at 135° C. for 4 hours and then poured on 500 mL water at 0° C. The organic phase is separated and washed with water three times. Most of the remaining capronic acid is distilled off. The crude product is titrated with toluene and petrol ethers until the product crystallizes out. The product is filtered and washed. Yield is 47%.

### EXAMPLE 4

### Synthesis of 4-hexylresorcinol

85 g 4-caproylresorcinol is dissolved in 770 mL of MeOH. 25 g Ni-catalyst are added and the mixture is hydrogenated at 70° C. and 10 bar. The GC quality of the crude material is 94%. The catalyst is filtered off, most of the MeOH is distilled off, 350 mL of toluene are added and the mixture is distilled until the head temperature indicates full removal of the MeOH. The mixture is cooled to room temperature, and the product is filtered off and dried.

### EXAMPLE 5

### Comparative

### Synthesis of 4-caproylresorcinol with HF

525 g resorcinol are dissolved in 2 kg 100% HF at 70-80° C. in an autoclave. At 70-80° C., 540 g capronic acid are added in portions. The mixture is heated for 10-15 hours at 70-80° C. The mixture is cooled to RT and quenched on 2.7 kg water. 3 l toluene are added and a phase separation is conducted at 40-50° C. The organic phase is washed repeatedly with water, bicarbonate solution and brine, dried azeotropically to yield, after crystallization, 60% product.

### EXAMPLE 6

### Comparative

### Synthesis of 4-hexylresorcinol with 4-caproylresorcinol Derived from the HF Process

85 g 4-caproylresorcinol (derived from the Example 5, HF-process) is dissolved in 255 mL of MeOH. 10 g Ni-catalyst are added and the mixture is hydrogenated at 80-95° C. and 30-50 bar. The GC quality of the crude material is only 86%. The catalyst is filtered off, most of the MeOH is distilled off, 350 mL of toluene are added and the mixture is distilled until the head temperature indicates full removal of the MeOH. The mixture is cooled to room temperature, and the product is filtered off and dried.

While the present invention has been particularly shown and described with reference to preferred embodiments, it will be readily appreciated by those of ordinary skill in the art that various changes and modifications may be made without departing from the spirit and scope of the invention. It is intended that the claims be interpreted to cover the disclosed embodiment, those alternatives which have been discussed above and all equivalents thereto.

What is claimed is:

1. A process for the production of 4-(C₂-C₁₆)alkylresorcinol which comprises reacting resorcinol with a (C₂-C₁₆)alkyloic acid in the presence of a zinc chloride catalyst to produce an intermediate comprising a 4-(C₂-C₁₆)acylresorcinol and zinc ions; adjusting the intermediate such that the zinc ion content is from about 0.1 to about 150 parts per million based on the weight of the intermediate to form an adjusted intermediate; and hydrogenating the produced 4-(C₂-C₁₆)acylresorcinol in the presence of the zinc ions and a base metal hydrogenation catalyst to produce a crude product comprising 4-(C₂-C₁₆)alkylresorcinol.

2. The process of claim 1 further comprising the subsequent step of isolating the 4-(C₂-C₁₆)alkylresorcinol from the crude product.

3. The process of claim 1 further comprising the subsequent step of isolating the 4-(C₂-C₁₆)alkylresorcinol from the crude product by one or more steps selected from the group consisting of filtering, distilling, washing, crystallizing, and drying.

4. The process of claim 1 wherein the (C₂-C₁₆)alkyloic acid comprises propionic acid.

5. The process of claim 1 wherein the (C₂-C₁₆)alkyloic acid comprises hexanoic acid.

6. The process of claim 1 wherein the base metal hydrogenation catalyst comprises nickel, cobalt, copper or combinations thereof.

7. The process of claim 1 wherein the base metal catalyst comprises nickel.

8. The process of claim 1 wherein the resorcinol is reacted with the (C₂-C₁₆)alkyloic acid at a temperature of from about 90° C. to about 125° C. for from about 3 hours to about 10 hours.

9. The process of claim 1 wherein the resorcinol is reacted with the (C₂-C₁₆)alkyloic acid at a temperature of from about 110° C. to about 125° C. for from about 3 hours to about 5 hours.

10. The process of claim 1 wherein the resorcinol is reacted with the (C₂-C₁₆)alkyloic acid at a temperature of from about 117° C. to about 120° C. for from about 3 hours to about 4 hours.

11. The process of claim 1 wherein the zinc ion content is adjusted by washing and crystallization.

12. The process of claim 1 wherein the hydrogenating is conducted at a temperature of from about 20° C. to about 120° C. at a pressure of from about 1 bar to about 50 bar for from about 1 hour to about 10 hours.

13. The process of claim 1 wherein the hydrogenating is conducted at a temperature of from about 50° C. to about 90° C. at a pressure of from about 1 bar to about 30 bar for from about 1 hour to about 5 hours.

14. The process of claim 1 wherein the hydrogenating is conducted at a temperature of from about 60° C. to about 80° C. at a pressure of from about 2 bar to about 20 bar for from about 1 hour to about 3 hours.

15. The process of claim 1 wherein the crude product comprises 4-(C₂-C₁₆)alkylresorcinol at a purity of at least about 92%.

16. The process of claim 1 wherein the crude product comprises 4-(C₂-C₁₆)alkylresorcinol at a purity of from about 92% to about 95%.

17. The process of claim 1 wherein the (C₂-C₁₆)alkyloic acid is reacted with the resorcinol at a temperature of from about 90° C. to about 125° C. for from about 3 hours to about 10 hours; wherein the zinc ion content is adjusted by washing and crystallization; wherein the hydrogenating is conducted at a temperature of from about 20° C. to about 120° C. at a pressure of from about 1 bar to about 50 bar for from about 1 hour to about 10 hours to obtain a crude product having a 4-alkylresorcinol purity of at least about 92%; and then isolating the 4-alkylresorcinol from the crude product by one or more steps selected from the group consisting of filtering, distilling, washing, crystallizing and drying.

18. The process of claim 1 wherein the (C₂-C₁₆)alkyloic acid is reacted with the resorcinol at a temperature of from about 117° C. to about 120° C. for from about 3 hours to about 4 hours; wherein the zinc ion content is adjusted by washing and crystallization; wherein the hydrogenating is conducted at a temperature of from about 60° C. to about 80° C. at a pressure of from about 2 bar to about 20 bar for from about 1 hour to about 3 hours to obtain a crude product having a 4-alkylresorcinol purity of from about 92% to about 95%; and then isolating the 4-alkylresorcinol from the crude product by one or more steps selected from the group consisting of filtering, distilling, washing, crystallizing and drying.

19. The process of claim 1 wherein the (C₂-C₁₆)alkyloic acid comprises propionic acid; the resorcinol is reacted with the propionic acid at a temperature of from about 117° C. to about 120° C. for from about 3 hours to about 4 hours; wherein the zinc ion content is adjusted by washing and crystallization; wherein the hydrogenating is conducted with a nickel hydrogenation catalyst at a temperature of from about 60° C. to about 80° C. at a pressure of from about 2 bar to about 20 for from about 1 hour to about 3 hours to obtain crude product comprising 4-propylresorcinol at a purity of from about 92% to about 95%; and then isolating the 4-propylresorcinol from the crude product by one or more steps selected from the group consisting of filtering, distilling, washing, crystallizing and drying.

20. The process of claim 1 wherein the (C₂-C₁₆)alkyloic acid comprises hexanoic acid; the resorcinol is reacted with the hexanoic acid at a temperature of from about 117° C. to about 120° C. for from about 3 hours to about 4 hours; wherein the zinc ion content is adjusted by washing and crystallization; wherein the hydrogenating is conducted with a nickel hydrogenation catalyst at a temperature of from about 80° C. to about 80° C. at a pressure of from about 2 bar to about 20 bar for from about 1 hour to about 3 hours to obtain a crude product comprising 4-hexyl-resorcinol with a purity of from about 92 to about 95%; and then isolating 4-hexylresorcinol from the crude product by one or more steps selected from the group consisting of filtering, distilling, washing, crystallizing and drying.
